(19) United States
(12) Patent Application Publication
(10) Pub. No.: US 2023/0295729 A1
(43) Pub. Date: Sep. 21, 2023

(54) METHODS FOR DETECTING AND TREATING PROGESTERONE-RESISTANT INFERTILITY

(71) Applicant: The Feinstein Institutes for Medical Research, Manhasset, NY (US)

(72) Inventors: Christine Metz, Great Neck, NY (US); Peter K. Gregersen, Larchmont, NY (US)

(21) Appl. No.: 18/120,714

(22) Filed: Mar. 13, 2023

Related U.S. Application Data
(60) Provisional application No. 63/320,290, filed on Mar. 16, 2022.

(51) Int. Cl. C12Q 1/6883; G16B 40/10; A61K 31/57

(52) U.S. Cl. CPC C12Q 1/6883; G16B 40/10; A61K 31/57; C12Q 2600/158

(57) ABSTRACT

Methods of treating and noninvasively detecting progesterone/progestin-resistant infertility are provided. Methods of identifying subjects with infertility as suitable for plant flavonoid treatments are provided.

### CROSS-REFERENCE TO RELATED APPLICATIONS

This application claims benefit of U.S. Provisional Application No. 63/320,290, filed Mar. 16, 2022, the contents of which are hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

Endometriosis is a complex disorder characterized by progesterone resistance. Roughly 30-50% of endometriosis patients experience infertility. The cause of endometriosis and its link to infertility is not completely understood. It has been shown that endometrial stromal cells (eSCs) from patients with endometriosis show decreased decidualization, a progesterone-driven process critical for implantation.

Thus, it is critically important to find methods and means for accurate and early diagnosis of progesterone resistant infertility in endometriosis (and also in subjects not experiencing endometriosis). In addition, to avoid unnecessary or incorrect intervention in this population, such as administering progestin in patients resistant to progestin, it is important to be able to distinguish between those who would benefit from progesterone/progestin treatments and those for whom such treatment will be of no benefit or a burden or who would need an adjunct treatment to benefit from the progesterone/progestin.

The present invention addresses these needs and provides methods of noninvasively detecting and treating progesterone resistance associated infertility in subjects with or without endometriosis.

### SUMMARY OF THE INVENTION

A method of treating infertility associated with progesterone-resistance in a female subject comprising administering to the subject an amount of a plant flavonoid or plant flavonoid analogue effective to increase fertility and/or reduce progesterone-resistance in the subject.

A method of reducing progression of or development of endometriosis in a female subject comprising administering to the subject an amount of a plant flavonoid or plant flavonoid analogue effective to reduce progression of, or development of, endometriosis in the subject.

A method of determining progesterone-resistance or progestin-resistance in a female subject comprising quantifying a response of menstrual effluent-derived endometrial stromal cells (eSCs) obtained from the subject to a progesterone (P4) or progesterone analogue, and comparing the response to a reference value.

A method of determining if an infertility in a female subject as suitable for treatment by a plant flavonoid or plant flavonoid analogue, comprising determining if the infertility is associated with progesterone-resistance in the subject by a method described herein, and, wherein the infertility is associated with progesterone-resistance, identifying the subject as suitable for treatment by a plant flavonoid or plant flavonoid analogue.

A method of determining if infertility in a female subject is suitable for treatment by a progesterone (P4) or progesterone analogue, comprising determining if the infertility is associated with progesterone-resistance in the subject by a method described herein, and, wherein the infertility is associated with progesterone-resistance, identifying the subject as suitable for treatment by a progesterone (P4) or progesterone analogue.

A method of determining an appropriate dosage amount of a progesterone (P4) or progesterone analogue for treating an infertility associated with progesterone-resistance in a female subject, comprising quantifying a level of progesterone-resistance in eSCs of the subject by a method described herein, comparing the quantified level with a reference value of appropriate dosage amounts for a range of progesterone-resistance levels, and selecting the appropriate dosage amount of a progesterone (P4) or progesterone analogue based on the quantified level.

A method of diagnosing an infertility in a female subject as associated with progesterone-resistance comprising quantifying a gene expression response of endometrial stromal cells (eSCs) obtained from the subject to a progesterone (P4) or progesterone analogue, and comparing the response to a reference value, so as to thereby diagnose an infertility in a female subject as associated with progesterone-resistance or as not associated with progesterone-resistance.

In embodiments, obtaining the eSCs comprises:
passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments;
treating the ME tissue fragments so as to disaggregate the tissue fragments into cells;
isolating the eSCs in the sample so treated. In embodiments, mutatis mutandis, fixation of fresh ME with PFA for further scRNA sequence analysis or qPCR analysis is performed.

In embodiments, the menstrual effluent (ME) sample is treated so as to enrich stromal cell content in the sample from 3%, or less, to 10%, or over, comprising: passing the sample of menstrual effluent (ME) through (i) a 70 μm pore filter or (ii) a filter that permits through passage of ME single cells but not of ME tissue fragments, so as to
separate ME tissue fragments from ME single cells;
collecting ME tissue fragments that have not passed through the filter;
enzymatically treating the ME tissue fragments so as to disaggregate the tissue fragments into cells; and
freezing the cells in methanol subsequent to disaggregating the tissue fragments,
wherein the preparation results in a stromal cell content in the sample of over 10%.

### BRIEF DESCRIPTION OF THE FIGURES

FIG. 1: Quercetin increased cAMP-induced decidualization as measured by the production of IGFBP1 and PRL production in eSCs obtained from controls and endometriosis cases.

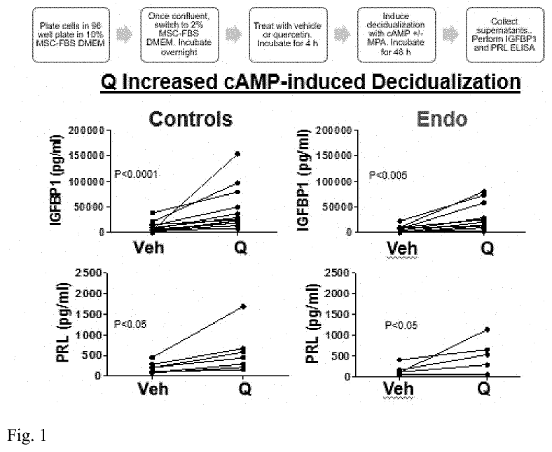

FIG. 2: Quercetin increased cAMP+MPA-induced decidualization, as measured by the production of IGFBP1 and PRL production in eSCs obtained from controls and endometriosis cases.

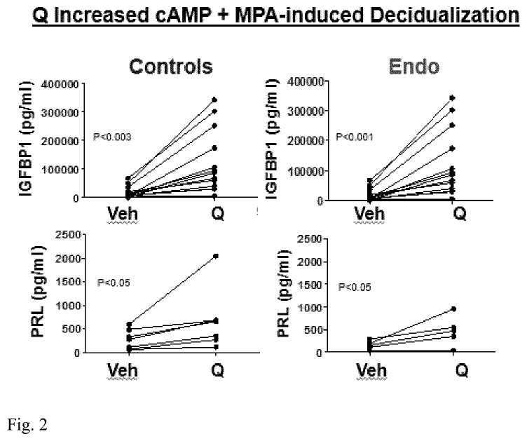

FIG. 3: Quercetin increases P4 target gene expression (HAND2 mRNA), in endometrial stroma cells obtained from healthy controls and endometriosis patients under vehicle-treated (upper panel) and cAMP+MPA treatment (lower panel).

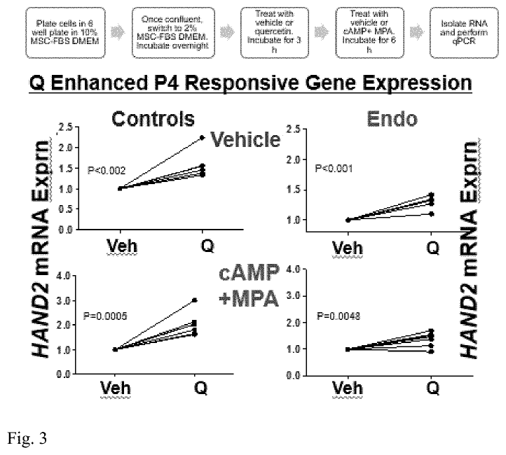

FIGS. 4A-4B: Quercetin-treatment of endometrial stromal cells reduces the senescence-associated secretory phenotype (SASP) and the expression SASP proteins. (4A) Treatment of confluent endometrial stromal cells with vehicle (Veh) vs. quercetin (25 μM, Q+) in DMEM 2% FBS for 2 days alters the expression of SASP proteins (or SASPs, IL-1β, TNF-membrane form [TNF-MF], TNF-soluble form [TNF-SF], MMP3, and Lamin B1 [LamB1*] vs. GAPDH [housekeeping protein for normalization]) by endometrial stromal cells, as determined by western blotting. Relative molecular weight markers (MWM) are shown on the left. (4B) Quantification of Veh-treated vs. Q-treated protein bands are shown in 4B. *Note: All proteins are positive SASPs (increased protein expression positively correlates with senescence), except LAMB1, which is lost with the senescence phenotype. Quercetin reduces senescence-associated secretory phenotype or SASP.

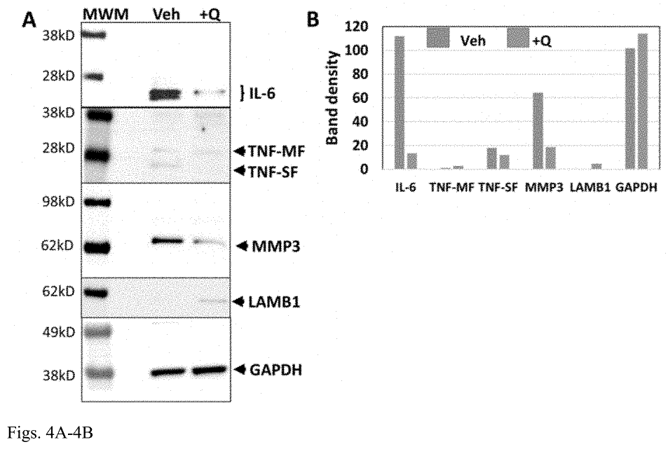

### DETAILED DESCRIPTION OF THE INVENTION

A method of treating infertility associated with progesterone-resistance in a female subject comprising administering to the subject an amount of a plant flavonoid or plant flavonoid analogue effective to increase fertility and/or reduce progesterone-resistance in the subject.

In embodiments, the methods further comprise identifying the subject as, or having the subject identified as, progesterone-resistant prior to treatment. In embodiments, the methods further comprise identifying the subject as progesterone-resistant prior to treatment. In embodiments, the methods further comprise having the subject identified as progesterone-resistant prior to treatment.

In embodiments, progesterone resistance in a subject is identified by quantifying a response of endometrial stromal cells (eSCs) from the subject to progesterone (P4) or a progesterone analogue.

In embodiments, the response quantified comprises gene expression.

In embodiments, the response quantified comprises PLZF and/or IHH gene expression.

In embodiments, the response quantified comprises CEBPD, DKK1, FABP4, MGP, and/or NNMT gene expression and/or IGFBP5, MALAT1, DIO2, and KRT17 gene expression.

In embodiments, the response quantified comprises gene expression determined by qPCR and/or single cell RNA-sequencing or protein expression

In embodiments, the progesterone analogue medroxyprogesterone acetate (MPA) or a progestogen or a progestin is used.

In embodiments, treating infertility associated with progesterone-resistance is indicated when the PLZF and/or IHH gene expression are less than a reference value for PLZF and/or IHH gene expression, respectively, for non-progesterone-resistant eSCs.

In embodiments, treating infertility associated with progesterone-resistance is indicated when the gene expression of CEBPD, DKK1, FABP4, MGP, and/or NNMT is less than a reference value for CEBPD, DKK1, FABP4, MGP, and/or NNMT gene expression, respectively, for non-progesterone-resistant eSCs.

In embodiments, treating infertility associated with progesterone-resistance is indicated when the gene expression of IGFBP5, MALAT1, DIO2, and/or KRT17 is greater than a reference value for IGFBP5, MALAT1, DIO2, and/or KRT17 gene expression, respectively, for non-progesterone-resistant eSCs.

In embodiments, eSCs are obtained from a menses/menstrual effluent sample from the subject.

In embodiments, the plant flavonoid is a flavonol having a 3-hydroxyflavone backbone.

In embodiments, the plant flavonoid is a quercetin or a fisetin. In embodiments, the plant flavonoid is a quercetin. In embodiments, the plant flavonoid analogue is a quercetin analogue. In embodiments, the plant flavonoid is administered as a pharmaceutical composition. In embodiments, the plant flavonoid is administered as an oral formulation or as a vaginal suppository.

In embodiments, the plant flavonoid or plant flavonoid analogue is administered at the time of ovulation in the subject.

In embodiments, the plant flavonoid or plant flavonoid analogue is not administered to the subject prior to ovulation and not after 7 days post-ovulation.

In embodiments, the time of ovulation is determined as within 24 hours of the subject's basal body temperature rising.

In embodiments, the time of ovulation is determined as within 24 hours after peak rising level of luteinizing hormone (LH) in the subject's urine.

In embodiments, the subject has, or has been diagnosed with, endometriosis.

In embodiments, the subject is also administered an amount of a progesterone (P4) or progesterone analogue to treat said infertility and the amount of a plant flavonoid or plant flavonoid analogue is administered prior to the administration of amount of a progesterone (P4) or progesterone analog.

A method is provided of reducing progression of or development of endometriosis in a female subject comprising administering to the subject an amount of a plant flavonoid or plant flavonoid analogue effective to reduce progression of, or development of, endometriosis in the subject.

In embodiments, the subject is an adolescent.

In embodiments, the subject is an adult.

A method is provided of determining progesterone-resistance or progestin-resistance in a female subject comprising quantifying a response of menstrual effluent-derived endometrial stromal cells (eSCs) obtained from the subject to a progesterone (P4) or progesterone analogue, and comparing the response to a reference value.

In embodiments, the subject is suspected of having, or diagnosed with, progesterone-resistance associated infertility.

In embodiments, the subject has endometriosis.

In embodiments, the response quantified comprises gene expression.

In embodiments, the response quantified comprises PLZF and/or IHH gene expression.

In embodiments, the response quantified comprises CEBPD, DKK1, FABP4, MGP, and/or NNMT gene expression and/or IGFBP5, MALAT1, DIO2, and KRT17 gene expression.

In embodiments, progesterone-resistance is indicated when the PLZF and/or IHH gene expression are less than a reference value for PLZF and/or IHH gene expression, respectively, for non-progesterone-resistant eSCs.

In embodiments, progesterone-resistance is indicated when the CEBPD, DKK1, FABP4, MGP, and/or NNMT gene expression are less than a reference value for CEBPD, DKK1, FABP4, MGP, and/or NNMT gene expression, respectively, for non-progesterone-resistant eSCs.

In embodiments, progesterone-resistance is indicated when the IGFBP5, MALAT1, DIO2, and KRT17 gene expression are more than a reference value for IGFBP5, MALAT1, DIO2, and KRT17 gene expression, respectively, for non-progesterone-resistant eSCs.

In embodiments, the response quantified comprises gene expression determined by qPCR and/or single cell RNA-sequencing, or by protein expression methods

In embodiments, the progesterone analogue medroxyprogesterone acetate (MPA) or a progestogen or a progestin is used.

In embodiments, eSCs are obtained from a menses sample from the subject.

In embodiments, the degree of progesterone-resistance is determined by comparing the quantified response to a control progesterone response curve for eSCs and identifying the degree of progesterone-resistance therefrom.

A method is provided of determining if an infertility in a female subject as suitable for treatment by a plant flavonoid or plant flavonoid analogue, comprising determining if the infertility is associated with progesterone-resistance in the subject by a method described herein, and, wherein the infertility is associated with progesterone-resistance, identifying the subject as suitable for treatment by a plant flavonoid or plant flavonoid analogue.

In embodiments, the method further comprises contacting eSCs from the subject with an amount of the plant flavonoid or plant flavonoid analogue and determining if progesterone-resistance is ameliorated thereby.

In embodiments, progesterone-resistance is ameliorated if decidualization of the eSCs in response to a progesterone (P4) or progesterone analogue (with or without cAMP) is increased by the plant flavonoid or plant flavonoid analogue.

In embodiments, the plant flavonoid is a flavonol having a 3-hydroxyflavone backbone.

In embodiments, the plant flavonoid is a quercetin or a fisetin.

In embodiments, the methods further comprise administering an amount of the plant flavonoid or plant flavonoid analogue to the subject effective to treat the infertility.

A method is provided of determining if an infertility in a female subject is suitable for treatment by a progesterone (P4) or progesterone analogue, comprising determining if the infertility is associated with progesterone-resistance in the subject by a method described herein, and, wherein the infertility is associated with progesterone-resistance, identifying the subject as suitable for treatment by a progesterone (P4) or progesterone analogue.

A method is provided of determining an appropriate dosage amount of a progesterone (P4) or progesterone analogue for treating an infertility associated with progesterone-resistance in a female subject, comprising quantifying a level of progesterone-resistance in eSCs of the subject by a method described herein, comparing the quantified level with a reference value of appropriate dosage amounts for a range of progesterone-resistance levels, and selecting the appropriate dosage amount of a progesterone (P4) or progesterone analogue based on the quantified level.

In embodiments, the methods further comprise administering the selected dosage amount of progesterone (P4) or progesterone analogue to the subject so as to treat the infertility.

In embodiments, the progesterone analogue is progestin.

In embodiments, the amount of progesterone or progesterone analogue administered comprises 80-120 mg once or twice per day.

In embodiments, the amount of progesterone or progesterone analogue administered comprises less than 80 mg once or twice per day.

In embodiments, the amount of progesterone or progesterone analogue is administered intravaginally.

In embodiments, the amount of progesterone or progesterone analogue is administered orally or intramuscularly.

In embodiments, the appropriate dosage amount of a progesterone (P4) or progesterone analogue based on the quantified level is 80-120 mg once or twice per day.

In embodiments, the appropriate dosage amount of a progesterone (P4) or progesterone analogue based on the quantified level is less than 80 mg once or twice per day.

A method is provided of diagnosing an infertility in a female subject as associated with progesterone-resistance comprising quantifying a gene expression response of endometrial stromal cells (eSCs) obtained from the subject to a progesterone (P4) or progesterone analogue, and comparing the response to a reference value, so as to thereby diagnose an infertility in a female subject as associated with progesterone-resistance or as not associated with progesterone-resistance.

In embodiments, the response quantified comprises PLZF and/or IHH gene expression.

In embodiments, the response quantified comprises CEBPD, DKK1, FABP4, MGP, and/or NNMT gene expression and/or IGFBP5, MALAT1, DIO2, and KRT17 gene expression.

In embodiments, progesterone-resistance is indicated when the PLZF and/or IHH gene expression are less than a reference value for PLZF and/or IHH gene expression, respectively, for non-progesterone-resistant eSCs.

In embodiments, progesterone-resistance is indicated when the CEBPD, DKK1, FABP4, MGP, and/or NNMT gene expression are less than a reference value for CEBPD, DKK1, FABP4, MGP, and/or NNMT gene expression, respectively, for non-progesterone-resistant eSCs.

In embodiments, progesterone-resistance is indicated when the IGFBP5, MALAT1, DIO2, and KRT17 gene expression are more than a reference value for IGFBP5, MALAT1, DIO2, and KRT17 gene expression, respectively, for non-progesterone-resistant eSCs.

In embodiments, the response quantified comprises gene expression determined by qPCR and/or single cell RNA-sequencing or by quantitative protein measurements.

In embodiments, the progesterone analogue medroxyprogesterone acetate (MPA) or a progestogen or a progestin is used.

In embodiments, the methods further comprise treating the subject identified as having an infertility associated with progesterone-resistance with an amount of a plant flavonoid or plant flavonoid analogue effective to increase fertility and/or reduce progesterone-resistance in the subject.

In embodiments, the methods further comprise treating the subject identified as having an infertility not associated with progesterone-resistance with an amount of a progesterone or progesterone analogue effective to increase fertility in the subject.

In embodiments, the eSCs are obtained from menstrual effluent of the subject.

In embodiments, the subject has endometriosis

In embodiments, the methods further comprise enriching the sample for eSCs by removing CD45+ cells from the sample prior to performing (i) qPCR gene expression analysis or (ii) scRNA-seq analysis, or (iii) protein analysis.

In embodiments, the methods further comprise depleting epithelial cells from the sample prior to performing (i) qPCR gene expression analysis or (ii) scRNA-seq analysis. In embodiments, epithelial cells are removed by a short adhesion step or by using depletion with anti-CD325/EpCAM.

In embodiments, fixation of fresh ME with PFA for further scRNA sequence analysis or qPCR analysis is performed; or treating the ME tissue fragments so as to disaggregate the tissue fragments into cells comprises contacting the ME tissue fragments with a collagenase. In embodiments, the collagenase is a collagenase I. In further embodiments, the tissue fragments are treated with a DNase. In embodiments, a collagenase I (1 mg/ml)/DNase (0.5 mg/ml) mixture is used on the tissue for 15 min/37° C. In embodiments, the resultant released cells are subjected to RBC lysis, neutrophil depletion and Ficoll centrifugation to remove dead cells before freezing in PFA or methanol for scRNA seq analysis or qPCR.

In embodiments, the methods further comprise freezing the cells in methanol subsequent to disaggregating the tissue fragments.

In embodiments, the methods further comprise one or more of:
lysing red blood cells in the sample;
depleting neutrophils from the sample; and
removing dead cells from the sample;
prior to performing (i) qPCR gene expression analysis or (ii) scRNA-seq analysis.

In embodiments, the obtained eSCs are maintained in culture prior to contacting them with a progesterone, progesterone analogue, plant flavonoid or plant flavonoid analogue.

In embodiments, the methods further comprise passing the ME tissue fragments separated from the ME single cells through a second filter having a 40 μm pore diameter and wherein the collecting the ME tissue fragments is performed on the ME tissue fragments that do not pass through the second filter.

In embodiments, the sample has been collected in a menstrual cup or a menstrual sponge.

In embodiments, the methods further comprise separating the eSCs using surface markers prior to performing (i) qPCR gene expression analysis or (ii) scRNA-seq analysis on the cells or (iii) protein analysis.s.

In embodiments, separation is effected using fluorescence-activated cell sorting or magnetic-activated cell sorting.

In embodiments, the subject is a human. In embodiments the subject is pre-menopausal. In embodiments, the subject is an adolescent. In embodiments, the adolescent is 12 years to <18 years.

In embodiments of the methods the stromal cells are CD45−/CD326−/CD31-/CD90+/CD105+/CD73+. In embodiments of the methods the stromal cells are CD140b+.

In embodiments of the methods the stromal cells exhibit a phenotype, or gene expression pattern, associated with endometriosis, wherein the phenotype or gene expression pattern is a pro-inflammatory or a senescent phenotype or gene expression pattern.

Menstrual cups as described herein include, but are not limited to, those sold by Diva International Inc., Ontario, Canada. Sponges for collecting ME as discussed herein include, but are not limited to, polyether polyurethane menstrual sponges. In addition other cotton based collection may be used.

In embodiments of the methods herein involving administration of a plant flavonoid, the plant flavonoid can be a quercetin or a fisetin. In embodiments, the plant flavonoid is a quercetin. In embodiments, the plant flavonoid analogue is a quercetin analogue. In embodiments, the plant flavonoid is administered as a pharmaceutical composition. In embodiments, the plant flavonoid is administered as an oral formulation or as a vaginal suppository.

As used herein, a control value is a value decided or obtained, usually beforehand, as a control. The concept of a control is well-established in the field, and can be determined, in a non-limiting example, empirically from non-afflicted subjects (versus afflicted subjects, including afflicted subjects having different grades of the relevant affliction such as progesterone-resistant infertility and/or endometriosis), and may be normalized as desired (in non-limiting examples, for volume, mass, age, location, gender) to negate the effect of one or more variables.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

Quercetin is a flavonoid with anti-inflammatory and immunomodulatory activities. In a rat model of endometriosis, quercetin inhibits the growth of endometrial implants and exerted anti-inflammatory effects. In addition, quercetin improves infertility in patients with polycystic ovary syndrome (PCOS). We assessed the effect of quercetin on decidualization and progesterone sensitivity using eSCs.

### Example 1

Methods: eSCs were isolated from menstrual effluent (ME) obtained from healthy controls and surgically diagnosed endometriosis cases. To induce decidualization, passage 2-3 eSCs DMEM 2% MSC-FBS were treated with dibutryl-cAMP (cAMP, 0.5 mM)±10-7 M medroxyprogesterone acetate (MPA) for 48 hr. eSCs were pre-treated with vehicle or quercetin (25 μM) 4 hr prior to decidualization. PRL and IGFBP1 protein and mRNA in the culture supernatants and cells, respectively, were assessed by ELISA and RT-qPCR, respectively. Endometrial stromal cells (eSCs) were isolated and grown in DMEM 10% MSC-FBS, PSQ, normocin. Passage 2-3 eSCs were split into 96 well plates or 6-well plates in DMEM 10% MSC-FBS. After overnight culture in DMEM 2% MSC-FBS, decidualization of eSCs was induced after exposure to vehicle or 25 μM quercetin for 4 hrs, followed by 0.5 mM cAMP±0.1 μM MPA for 48 hrs. Decidualization was assessed by measuring IGFBP1 and/or PRL levels in the culture supernatants by ELISA. Progesterone sensitivity was also assessed by measuring progesterone-induced genes. Two sample t tests (unpaired or paired) were used to determine significance. Assessment of progesterone sensitive genes eSCs were treated vehicle or 25 μM quercetin for 4h followed by veh or 0.5 mM cAMP+0.1 uM MPA for 6h; RNA was isolated; and qPCR was performed for the P4 sensitives gene: HAND2, or other P4 target genes.

Results: Quercetin significantly enhanced cAMP-induced decidualization of control-eSCs (p<0.02) and case-eSCs (p<0.05), although eSCs from cases showed reduced decidualization. Addition of MPA potentiated cAMP-induced decidualization in control-eSCs, and, to a lesser extent case-eSCs and this was significantly enhanced by pre-treating with quercetin (p<0.05). When quercetin alone was added to control- and case-eSCs, MPA-induced HAND2 mRNA expression was significantly increased over vehicle (p<0.05). Interestingly, ibuprofen and dexamethasone as tested in the same system did not potentiate decidualization, neither did N-acetylcysteine (NAC), an antioxidant. SA (salicylic acid) also did not potentiate decidualization.

Conclusion: Endometriosis-eSCs exhibit decreased decidualization compared to control-eSCs. MPA potentiates decidualization when added with cAMP and quercetin further enhances control-eSC and case-eSC decidualization and MPA responsiveness. Accordingly, 3-hydroxyflavone (flavanols), including quercetin, can enhance progesterone sensitivity in endometriosis.

### Example 2

Quercetin Enhances Decidualization:

Roughly 30-50% of endometriosis patient experience infertility. The cause of endometriosis and its link to infertility is not completely understood. It has been shown that endometrial stromal cells (eSCs) from patients with endometriosis show decreased decidualization, a progesterone-driven process critical for implantation. Quercetin is a flavonoid with anti-inflammatory and immunomodulatory activities. In a rat model of endometriosis, quercetin inhibited the growth of endometrial implants and exerted anti-inflammatory effects. In addition, quercetin improves infertility in patients with polycystic ovary syndrome (PCOS). We assessed the effect of quercetin on decidualization and progesterone sensitivity using eSCs. Methods: eSCs were isolated from menstrual effluent (ME) obtained from healthy controls and surgically diagnosed endometriosis cases. To induce decidualization, passage 2-3 eSCs were treated with dibutryl-cAMP (cAMP, 0.25-0.5 mM)±10-7M medroxyprogesterone acetate (MPA) for 48 hr. eSCs were pre-treated with vehicle or quercetin (25 μM) 4 hr prior to decidualization. PRL and IGFBP1 protein and mRNA in the culture supernatants and cells, respectively, were assessed by ELISA and RT-qPCR, respectively. Progesterone sensitivity was also assessed by measuring progesterone-induced genes, PLZF and IHH. Two sample t tests (unpaired or paired) were used to determine significance.

Results: Quercetin significantly enhanced cAMP-induced decidualization of control-eSCs (p<0.02) and case-eSCs (p<0.05), although eSCs from cases showed reduced decidualization. Addition of MPA potentiated cAMP-induced decidualization in control-eSCs, and, to a lesser extent case-eSCs and this was significantly enhanced by pre-treating with quercetin (p<0.05). When quercetin alone was added to control and case-eSCs, MPA-induced PLZF and IHH mRNA expression was significantly increased over vehicle (p<0.05).

Conclusion: Endometriosis-eSCs exhibit decreased decidualization compared to control-eSCs. MPA potentiates decidualization when added with cAMP and quercetin further enhances control-eSC and case-eSC decidualization and MPA responsiveness.

### Example 3

Endometriosis eSCs exhibit reduced progesterone-sensitivity Endometriosis is a disorder that affects ˜10% of females of reproductive age. Decidualization, a progesterone (P4) driven process in which endometrial stromal cells (eSCs) differentiate to support implantation, is impaired in endometriosis. It is proposed that these P4-induced changes are mediated by the transcriptional activity of its canonical receptors (PR-A and PR-B). While prior studies focused on P4 resistance in ectopic endometriosis lesions, we sought to investigate P4 responsiveness using eutopic eSCs obtained from endometriosis cases and controls.

Methods: Low passage (p2-3) menstrual effluent-derived eSCs (ME-eSCs) obtained from women with confirmed endometriosis or healthy controls were stimulated with vehicle or MPA, a stable P4 analogue. qPCR and single cell RNA-seq (scRNA-seq, n=4 controls and 4 cases) were used to measure P4-induced gene expression and identify dysregulated pathways. Subcellular localization of PR isoforms and the PR chaperone protein, FKBP52, was assessed using immunofluorescent microscopy and western blotting of total, nuclear, and cytoplasmic preparations of ME-eSCs±MPA stimulation.

Results: MPA-stimulated PLZF and IHH gene expression are significantly blunted in ME-eSCs from cases vs. controls, as determined by qPCR. At baseline, global gene expression by scRNA-Seq of control ME-eSCs significantly differed from case ME-eSCs; MPA stimulation induced differentially expressed genes, including CEBPD, DKK1, FABP4, MGP, and NNMT which were significantly unregulated in controls only and IGFBP5, MALAT1, DIO2, and KRT17 that were significantly downregulated in controls only. PR localization studies support differential subcellular localization of PR isoforms and chaperone FKBP52 in cases and controls under baseline and MPA-stimulation conditions.

Conclusion: ME-derived eSCs provide a model system for studying eutopic P4 sensitivity. In endometriosis, eutopic ME-eSCs show significantly blunted responsiveness to P4 stimulation. This attenuated P4 response may be, in part, due to altered FKBP52 and PR expression and nuclear localization.

### REFERENCES

Zondervan K T, Becker C M, Koga K, Missmer S A, Taylor R N, Viganò P. Endometriosis. Nat Rev Dis Primer. 2018; 4(1):1-25
Gellersen B, Brosens I, Brosens J. Decidualization of the Human Endometrium: Mechanisms, Functions, and Clinical Perspectives. Semin Reprod Med. 2007; 25(6):445-453.
Nayyar A, Saleem M I, Yilmaz M, et al. Menstrual Effluent Provides a Novel Diagnostic Window on the Pathogenesis of Endometriosis. Front Reprod Health. 2020; 2. doi:10.3389/frph. 2020.00003
Jamali, N., F. Zal, Z. Mostafavi-Pour, M. Samare-Najaf, T. Poordast, and A. Dehghanian. 2021. “Ameliorative Effects of Quercetin and Metformin and Their Combination Against Experimental Endometriosis in Rats.” Reprod Sci 28 (3):683-692.
Deryabin P, Griukova A, Nikolsky N, Borodkina A. The link between endometrial stromal cell senescence and decidualization in female fertility: the art of balance. Cell Mol Life Sci. 2020; 77(7): 1357-1370.
Bhurke, A S, Bagchi, IC, Bagchi, MK. Progesterone-regulated endometrial factors controlling implantation. Am J Reprod Immunol 2016; 75: 237— 245.
Vaez S, Parivr K, Amidi F, Rudbari N H, Moini A, Amini N. Quercetin and polycystic ovary syndrome; inflammation, hormonal parameters and pregnancy outcome: A randomized clinical trial. Am J Reprod Immunol. 2023 March; 89(3): e13644.
Patel B G, Rudnicki M, Yu J, Shu Y, Taylor R N. Progesterone resistance in endometriosis: origins, consequences and interventions. Acta Obstet Gynecol Scand. 2017 June; 96(6):623-632. doi: 10.1111/aogs.13156.

1. A method of treating infertility associated with progesterone-resistance in a female subject comprising administering to the subject an amount of a plant flavonoid or plant flavonoid analogue effective to increase fertility and/or reduce progesterone-resistance in the subject.

2. The method of claim 1, further comprising identifying the subject as, or having the subject identified as, progesterone-resistant prior to treatment.

3. The method of claim 1, wherein progesterone resistance in a subject is identified by quantifying a response of endometrial stromal cells (eSCs) from the subject to progesterone (P4) or a progesterone analogue.

4. The method of claim 3, wherein the response quantified comprises gene expression.

5. The method of claim 3, wherein the response quantified comprises PLZF and/or IHH gene expression.

6. The method of claim 1, wherein the response quantified comprises CEBPD, DKK1, FABP4, MGP, and/or NNMT gene expression and/or IGFBP5, MALAT1, DIO2, and KRT17 gene expression.

7. The method of claim 1, wherein the response quantified comprises gene expression determined by qPCR and/or single cell RNA-sequencing.

8. The method of claim 2, wherein the progesterone analogue medroxyprogesterone acetate (MPA) or a progestogen or a progestin is used.

9. The method of claim 5, wherein treating infertility associated with progesterone-resistance is indicated when the PLZF and/or IHH gene expression are less than a reference value for PLZF and/or IHH gene expression, respectively, for non-progesterone-resistant eSCs.

10. The method of claim 6, wherein treating infertility associated with progesterone-resistance is indicated when the gene expression of CEBPD, DKK1, FABP4, MGP, and/or NNMT is less than a reference value for CEBPD, DKK1, FABP4, MGP, and/or NNMT gene expression, respectively, for non-progesterone-resistant eSCs.

11. The method of claim 8, wherein treating infertility associated with progesterone-resistance is indicated when the gene expression of IGFBP5, MALAT1, DIO2, and/or KRT17 is greater than a reference value for IGFBP5, MALAT1, DIO2, and/or KRT17 gene expression, respectively, for non-progesterone-resistant eSCs.

12. The method of claim 3, wherein eSCs are obtained from a menses/menstrual effluent sample from the subject.

13. The method of claim 1, wherein the plant flavonoid is a flavonol having a 3-hydroxyflavone backbone.

14. The method of claim 1, wherein the plant flavonoid is a quercetin or a fisetin.

15. The method of claim 1, wherein the plant flavonoid analogue is a quercetin analogue.

16. The method of claim 1, wherein the plant flavonoid or plant flavonoid analogue is administered at the time of ovulation in the subject.

17-20. (canceled)

21. The method of claim 1 wherein the subject is also administered an amount of a progesterone (P4) or progesterone analogue to treat said infertility and the amount of a plant flavonoid or plant flavonoid analogue is administered prior to the administration of amount of a progesterone (P4) or progesterone analog.

22. A method of reducing progression of or development of endometriosis in a female subject comprising administering to the subject an amount of a plant flavonoid or plant flavonoid analogue effective to reduce progression of, or development of, endometriosis in the subject.

23-24. (canceled)

25. A method of determining progesterone-resistance or progestin-resistance in a female subject comprising quantifying a response of menstrual effluent-derived endometrial stromal cells (eSCs) obtained from the subject to a progesterone (P4) or progesterone analogue, and comparing the response to a reference value.

26-37. (canceled)

38. A method of determining if an infertility in a female subject as suitable for treatment by a plant flavonoid or plant flavonoid analogue, comprising determining if the infertility is associated with progesterone-resistance in the subject by the method of claim 25, and, wherein the infertility is associated with progesterone-resistance, identifying the subject as suitable for treatment by a plant flavonoid or plant flavonoid analogue.

39-65. (canceled)
